(19) United States
(12) Patent Application Publication
(10) Pub. No.: US 2003/0120320 A1
(43) Pub. Date: Jun. 26, 2003

(54) Implantable medical device having a housing or component case with an insulating material formed thereon, and methods of making same

(75) Inventor: Warren Solom, Plymouth, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic,Inc.

(21) Appl. No.: 10/033,965

(22) Filed: Dec. 20, 2001

(51) Int. Cl. A61N 1/375

(52) U.S. Cl. 607/36

(57) ABSTRACT

An implantable medical device (IMD) comprising a device housing, and which may further include a component case. An insulating material or layer is formed on at least one of the interior and/or exterior surfaces of the device housing and/or component case. The insulating material may be comprised of a metal oxide, a plastic material, an epoxy material or a ceramic material.

### FIELD OF THE INVENTION

This invention relates generally to implantable medical devices, and, more particularly, to an implantable medical device having a housing or component case with an insulation layer formed thereon, and methods of making same.

### DESCRIPTION OF THE RELATED ART

Implantable medical devices for therapeutic stimulation of the heart are well known in the art. A cardiac pacemaker is one example of such a device. In general, a pacemaker delivers electric energy to the heart to initiate the depolarization of cardiac tissue. This stimulating regime is used to treat heart block by providing electrical stimulation in the absence of naturally occurring spontaneous cardiac depolarizations.

Another form of implantable medical device for therapeutic stimulation of the heart is an implantable defibrillator. Such defibrillator devices deliver energy to the heart to interrupt ventricular fibrillation of the heart. In operation, a defibrillator device detects ventricular fibrillation and delivers a high-voltage pulse to the heart through widely spaced electrodes located in or near the heart, thus mimicking transthoracic defibrillation.

Typically, such implantable medical devices are comprised of a variety of components that are assembled and positioned within a housing. For example, in the case of an implantable defibrillator, the components in the housing may include, among other things, an electronics module, a battery or electrochemical cell, and a capacitor module. In the case of a pacemaker device, the capacitor module may be omitted. In general, the various components are preferably sized such that the volume and weight of the overall implantable device are as small as possible.

In some cases, the components of the implantable medical device may need to be thermally, chemically or electrically isolated from one another and/or the housing of the device for various reasons. For example, in modern implantable devices, the various components of the devices, e.g., the battery or the capacitor module, may have a thin insulating liner positioned around the component to assist in achieving a desired degree of electrical isolation. Thereafter, the insulated component is positioned within a component case and positioned within the device housing adjacent any previously installed component. After proper inspection and testing, the housing is sealed and the device is ready for implantation in a patient.

The packaging techniques generally described above may be performed on an individual component basis as well as on the overall final packaging for the device. For example, an illustrative packaging process for a capacitor module 20 that will ultimately be positioned in a completed heart defibrillator unit will now be described with reference to FIG. 1. Depicted therein is a capacitor case bottom 10, an insulating liner base 12, a capacitor module 14, an insulating liner top 16 and a capacitor case top 18. In the depicted embodiment, the various components have a generally semicircular configuration, although the configuration of such components may vary a great deal depending upon the shape of the component. The capacitor case bottom 10 and capacitor case top 18 are typically comprised of a metal, e.g., aluminum. The insulating liner base 12 and the insulating liner top 16 are comprised of an insulating material, such as a plastic. In essence, the insulating liner base 12 and top 16 are sized and configured to be form-fitted around the capacitor module 14 and within component case bottom 10.

The components depicted in FIG. 1 may be assembled in accordance with the following process. Initially, the capacitor module 14 is positioned within an internal cavity 13 in the insulating liner base 12, and the insulating liner top 16 is positioned above the capacitor module 14 and into approximately mated engagement with the insulating liner base 12. Thereafter, tape may be wrapped around the insulating liner base 12 (having the capacitor module 14 positioned therein) and the insulating liner top 16 to secure the components together thereby defining an insulated capacitor assembly 15. Thereafter, the insulated capacitor assembly 15 is positioned in a cavity 11 defined in the capacitor case bottom 10, and the capacitor case top 18 is welded to the capacitor case bottom 10. Of course, those skilled in the art will recognize that the various components described above will have minimal openings (not shown) for electrical leads (not shown) coupled to the capacitor module 14 and other electrical components or devices within the housing of the implantable device. Such details have been omitted for purposes of clarity.

As stated previously, such a packaging technique may be applied to other components within an implantable device, e.g., a battery, and/or with respect to the overall housing of the device. That is, the housing for the completed device may be comprised of at least two portions, e.g., two halves, and an insulating liner may be positioned adjacent the interior surface of each of the housing portions. Thereafter, the individually assembled components, e.g., electronics module, battery, capacitor module, etc., may be positioned within the housing portions. Then, the housing portions may be hermetically sealed to one another.

However, the procedures outlined above with respect to achieving some degree of electrical isolation are problematic in some respects. For example, positioning the insulating liner around the desired components of the device is time-consuming and increases manufacturing costs. Moreover, during such processes, the insulating liner may be torn or become wrinkled, thereby necessitating repair of the device, all of which may lead to increased production cycles and added expense. What is desired is a device and method of packaging implantable medical devices to assist in providing the desired degree of electrical isolation of the various components of the medical device in a timely and efficient manner.

The present invention is directed to overcoming, or at least reducing the effects of, one or more of the problems described above.

### SUMMARY OF THE INVENTION

In general, the present invention is directed to various embodiments of an implantable medical device having a device housing and/or component case with an insulation material or layer formed thereon. In one illustrative embodiment, the device comprises a device housing and a component case, each of which have a plurality of interior surfaces, and an insulating material or layer formed on at least one of the interior surfaces of the device housing or the component case. In some embodiments, the insulating material is formed on at least some of the interior surfaces of both the device housing and component case. In even further embodiments, the insulating material or layer may be formed on all of the interior surfaces of the component case and/or the device housing. In additional embodiments, the insulating material may be formed on one or more of the exterior surfaces of the component case and/or the device housing. In even further embodiments, the insulating material may be formed on both the interior and/or exterior surfaces of both the device housing and/or the component case. The insulating material may be comprised of a metal oxide, e.g., aluminum oxide, titanium oxide, etc., a plastic material, an epoxy material, a ceramic material, or other suitable material.

The present invention is also directed to various methods for forming the inventive medical devices disclosed herein. In one embodiment, the method comprises providing at least one of a device housing and a component case, each of which have a plurality of interior surfaces, and forming an insulating material or layer on at least one of the interior surfaces of the device housing or the component case. In some embodiments, the method involves forming the insulating material on at least some of the interior and/or exterior surfaces of both the device housing and the component case. In even further embodiments, the method involves forming the insulating material or layer on all of the interior surfaces of the device housing and/or component case. In other embodiments, the method may involve forming the insulating material on at least one of the exterior surfaces of the component case and/or device housing. In some embodiments, the method involves forming an insulating material or layer comprised of a metal oxide, a plastic material, an epoxy material or a ceramic material. In yet further embodiments, the insulating material is comprised of a metal oxide that is formed by performing at least one oxidation process. The insulating material or layer may be comprised of a plastic, an epoxy, a ceramic material, or other suitable material, and it may be formed by a spraying, brushing, dipping, vapor deposit, or other appropriate process followed by, in some cases, a curing process.

### BRIEF DESCRIPTION OF THE DRAWINGS

The invention may be understood by reference to the following description taken in conjunction with the accompanying drawings, in which like reference numerals identify like elements, and in which:

FIG. 1 depicts an illustrative capacitor module packaged in accordance with a known prior art technique;

FIGS. 2A-2G depict various perspective views of an illustrative implantable medical device in accordance with one embodiment of the present invention;

FIGS. 3A-3C are views of an illustrative component case for an implantable medical device in accordance with one aspect of the present invention; and

FIGS. 4A-4B are cross-sectional views of an illustrative housing for an implantable medical device in accordance with one illustrative embodiment of the present invention.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE INVENTION

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The present invention will now be described with reference to the attached figures. The relative sizes of the various features and structures depicted in the drawings may be exaggerated or reduced as compared to the size of those features or structures on real-world devices. Moreover, for purposes of clarity, the devices depicted herein do not include all of the detailed components of a real-world implantable medical device. Nevertheless, the attached drawings are included to describe and explain illustrative examples of the present invention.

In general, the present invention is directed to an implantable medical device having a housing or component case with an insulating material formed thereon, and methods of making same. The insulating material is integral with the housing and/or component case in that it is formed on at least one of the interior surfaces and/or exterior surfaces of the component case and/or device housing. As will be readily apparent to those skilled in the art upon a complete reading of the present application, the present invention is applicable to a variety of implantable medical devices, including, but not limited to, defibrillators, pacemakers, etc. Moreover, the present invention may be employed with a variety of medical processes and techniques designed for therapeutic stimulation of a human heart.

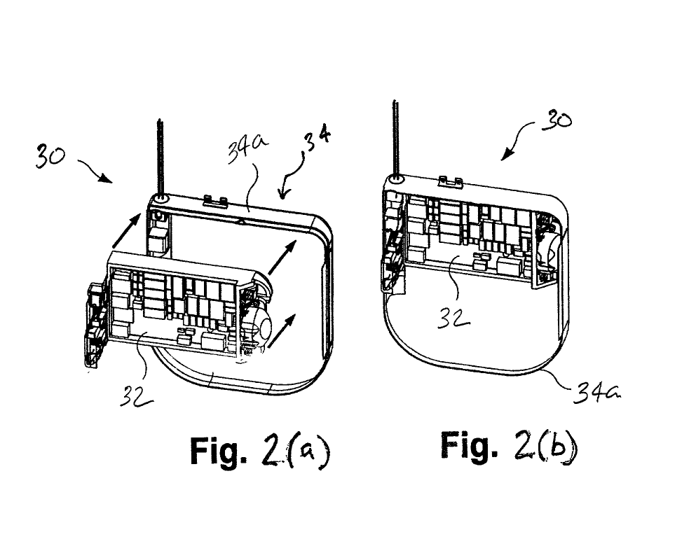

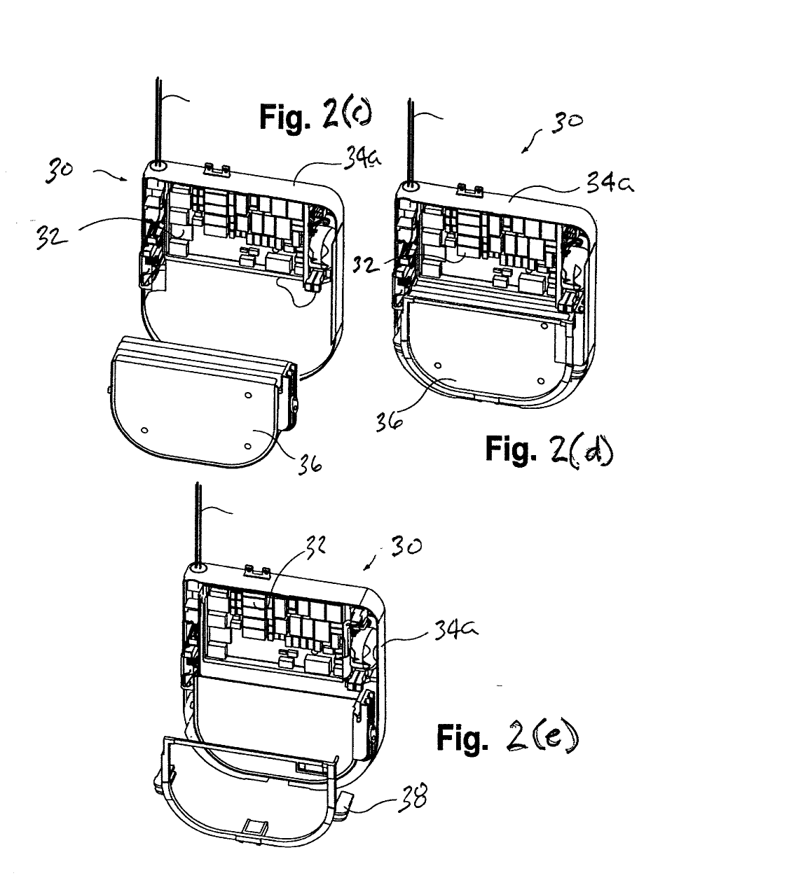

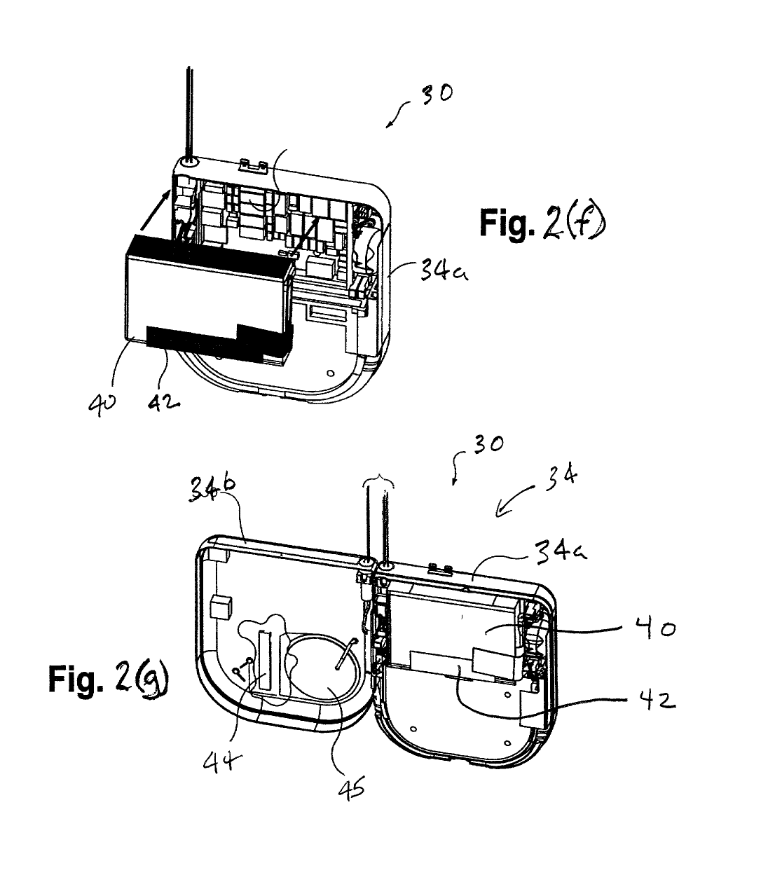

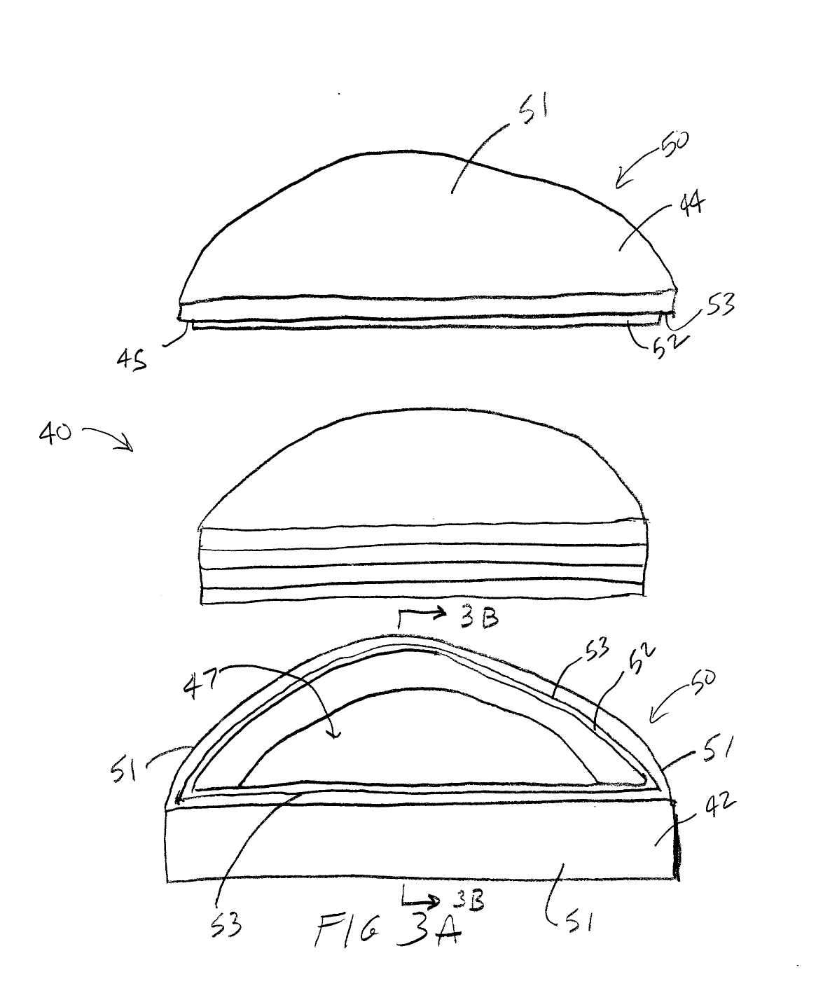

FIGS. 2A-2F depict the assembly of the major components of an illustrative implantable defibrillator 30 comprised of a housing 34 in accordance with one illustrative embodiment of the present invention. As shown in FIGS. 2A-2B, an electronics module 32 is positioned in one-half 34a of the housing 34. The electronics module 32 is comprised of various electrical circuitry needed for the implantable defibrillator 30 to perform its intended function. FIG. 2B depicts the electronics module 32 after it has been seated in the housing portion 34a. FIGS. 2C-2E depict the positioning of a capacitor module 36 in the housing portion 34a adjacent the electronics module 32. FIG. 2E depicts an insulator cup 38 that is positioned adjacent the capacitor module 36 to assist in electrically isolating the capacitor module 36 from other components of the device 30.

FIGS. 2F-2G depict a battery 40 positioned in the housing portion 34a. An insulating film 42 is positioned around a portion of the battery 40 to assist in electrically isolating the battery 40 from various components of the device 30. The battery 40 provides the electrical energy required to charge and re-charge the capacitors on the capacitor module 36 and to power the circuitry on the electronics module 32. FIG. 2G also depicts a second portion 34b of the housing 34 connected to the first portion 34a of the housing 34. An activity sensor 44 and a patient alert apparatus 45 are disposed in the lower part of the second portion 34b of the housing 34. The first and second portions 34a, 34b of the housing 34 are subsequently closed and hermetically sealed, although that is not depicted in the figures.

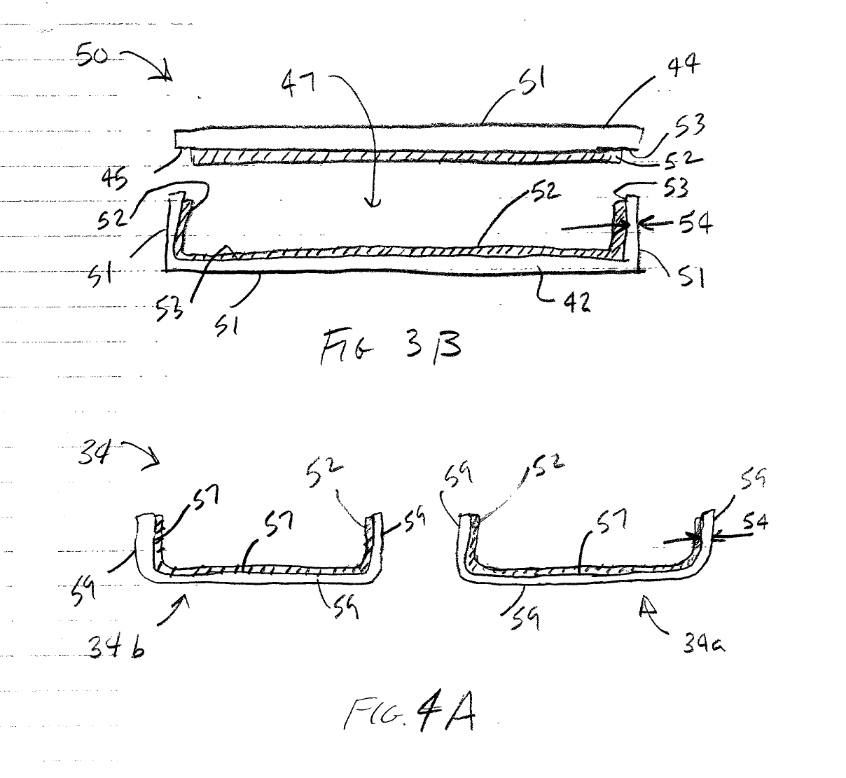

FIG. 3A depicts an illustrative component 40 that will be positioned within the housing 34 of the implantable device 30. The component 40 will be positioned within a component case 50 comprised of a component case bottom 42 and a component case top 44. The component case 50 is generally comprised of a plurality of exterior surfaces 51 and a plurality of interior surfaces 53. In one illustrative embodiment, as described more fully below, the present invention is directed to forming an layer of insulating material on at least one of the interior surfaces 53 and/or the exterior surfaces 51 of the component case 50.

As shown in FIGS. 3A-3B, an insulating material 52 is formed on the interior surfaces 53 of the component case bottom 42 and the bottom surface 45 of the component case top 44. The component case bottom 42 defines an interior cavity 47 adapted to receive the component 40. In the depicted embodiment, the component 40 is a capacitor module. However, after a complete reading of the present application, those skilled in the art will recognize that the component 40 is illustrative in nature, and that the component 40 may be any type of component typically used in an implantable medical device, e.g., a battery, a fuel cell, an electronics module, a capacitor module, etc. Thus, the present invention should not be considered as limited to any particular type of component of an implantable device unless such limitations are clearly set forth in the appended claims. Moreover, those skilled in the art will recognize after reading the present disclosure that the size and configuration of the component case 50 may vary depending upon a variety of factors, e.g., the shape of the basic component positioned therein.

When assembled, the component case bottom 42 and the component case top 44 define the component case 50 containing the component 40. As assembled, the component case 50 has a plurality of interior surfaces 53, e.g., the interior surfaces of the cavity 47 (including the bottom and sidewalls defining the cavity 47) as well as the bottom surface 45 of the component case top 44. After the component 40 is positioned in the component case bottom 42, the component case top 44 and component case bottom 42 may be welded together. Of course, the insulating material 52 will need to be configured or trimmed such that the component case bottom 42 and component case top 44 may be appropriately positioned to facilitate welding or otherwise assembling these components together.

FIG. 3C depicts an illustrative embodiment wherein the insulating material 52 is formed on the exterior surfaces 51 of the component case 50. Of course, if desired, the insulating material may be formed on one or more of the interior surfaces 53 and the exterior surfaces 51 of the component case 50. Furthermore, in some embodiments, the insulating material may be formed on both the interior surfaces 53 and exterior surfaces 51 of the component case 50.

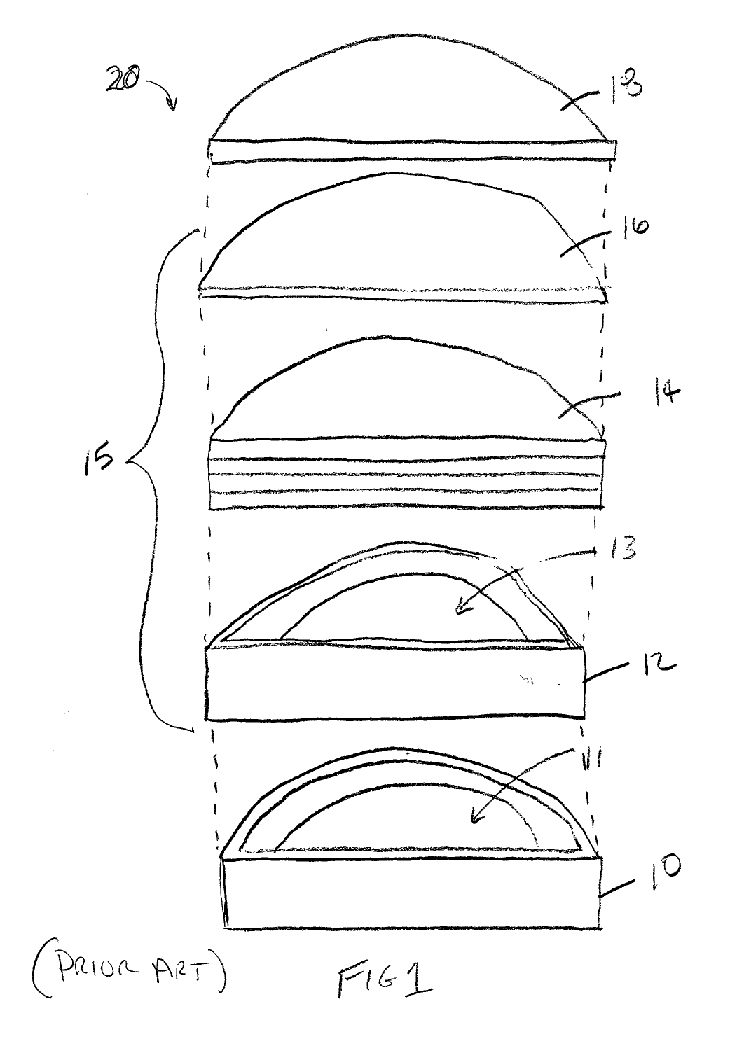

In another illustrative embodiment, as described more fully below, the present invention is also directed to forming a layer of insulating material on the interior surfaces and/or the exterior surfaces of the device housing 34. The formed insulating material 52 may be useful in providing the desired degree of electrical insulation of the components of the implantable device. Moreover, as will be appreciated after a complete reading of the present application, the present invention may be used in addition to, or in lieu of, the use of a separate insulating liner, such as the liner 12, 16 depicted in FIG. 1. The insulating material 52 is not depicted in FIGS. 2A-2G for purposes of clarity. As will be discussed more fully below, the insulating material 52 may be comprised of a variety of materials and it may be formed by a variety of techniques. Moreover, the thickness of the insulating material 52 may vary depending upon the specific application. For example, the thickness of the insulating material may range from approximately 10 nm to 500 μm.

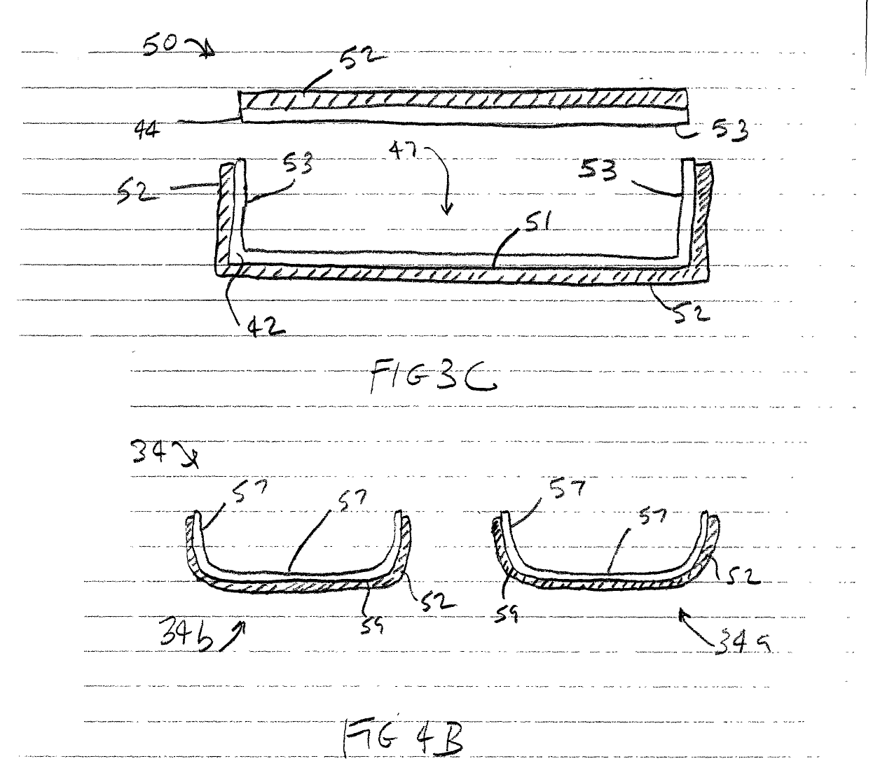

FIGS. 4A-4B depict an illustrative device housing 34 comprised of first and second portions 34a, 34b having a plurality of interior surfaces 57 and a plurality of exterior surfaces 59. The insulating material described herein may be formed on one or more of the interior surfaces 57 and/or exterior surfaces 59 of the housing 34. In the illustrative embodiment depicted in FIG. 4A, the insulating material 52 is formed on the interior surfaces 57 of the housing portions 34a, 34b. In the illustrative embodiment directed in FIG. 4B, the insulating material 52 is formed on the exterior surfaces 59 of the housing portions 34a, 34b. Of course, if desired, the insulating material may be formed on both the interior surfaces 57 and exterior surfaces 59 of both of the housing portions 34a, 34b.

The component case 50 and the housing portions 34a, 34b may be comprised of a variety of materials, e.g., aluminum, titanium, stainless steel, etc. The wall thickness 54 of the component case 50 and the housing portions 34a, 34b may be varied as a matter of design choice, e.g., the thickness 54 may range from approximately 0.002-0.030 inches for the component case 50 and approximately 0.002-0.030 inches for the housing portions 34a, 34b.

The insulating material 52 of the present invention may be comprised of a variety of materials and it may be formed on the interior surfaces and/or exterior surfaces of the component case 50 and/or the housing 34 by a variety of techniques. For example, the insulating material 52 may be comprised of a metal oxide, a plastic material, an epoxy material, a ceramic material, etc., that is formed on one or more of the interior surfaces and/or exterior surfaces of the component case 50 and/or housing 34.

In the case where the insulating material 52 is comprised of a metal oxide, an oxidation process is performed on the interior surfaces and/or exterior surfaces of the component case 50 and/or the housing portions 34a, 34b to form a metal oxide material 52 on the desired surfaces. Using this technique, the selection of the thickness 54 of the component case 50 and the housing portions 34a, 34b should allow for some consumption of the original thickness 54 during the process of forming the metal oxide material 52. The metal oxide material 52 will be an oxide of the material comprising the component case 50 and/or the housing portions 34a, 34b, e.g., aluminum oxide, titanium oxide, etc. The thickness of the metal oxide material 52 may be varied as a matter of design choice. In one illustrative embodiment, where the component case 50 is comprised of aluminum, the metal oxide material 52 is comprised of aluminum oxide.

The metal oxide material 52 may be formed by a variety of techniques. For example, in the illustrative example where the insulating material will be formed on the interior surfaces 53 of the component case 50, an appropriate oxidizing agent may be positioned in the internal cavity 47 of the component case bottom 42, electrodes may be coupled to the component case bottom 42, and the appropriate current may be passed through the component case bottom 42 and the oxidizing agent until such time as the metal oxide material 52 of the desired thickness is formed on the interior surfaces 53 of the component case bottom 42 in contact with the oxidizing agent. In a similar fashion, the bottom surface 45 of the component case top 44 may be oxidized.

The composition of the oxidizing agent may vary depending upon the materials of construction of the component case 50. In one illustrative embodiment, where the component case 50 is comprised of aluminum, the oxidizing agent may be any fluid, gas or charged field sufficient to accomplish the desired oxidation. In one illustrative embodiment, the oxidizing agent is an acid that is part of a liquid bath solution. Alternatively, the surfaces of the component case 50 on which the metal oxide material 52 is not desired may be taped or otherwise covered or masked. Thereafter, the component case 50 may be positioned in a bath comprised of the appropriate oxidizing agent to form the metal oxide material 52 on the exposed surfaces of the component case 50. As yet another alternative, the metal oxide material 52 may be formed by covering the surfaces of the component case 50 upon which the metal oxide material 52 is not desired, and then positioning the component case 50 in a furnace in an oxidizing ambient at the appropriate temperature to form the metal oxide material 52.

The insulating material 52 may also be comprised of an insulating plastic material, such as a spray-on product sold under the trade name Parylene® or a fluoroplastic (e.g., ETFE, PTFE, ECTFE, PCTFE, FEP, PFA or PVDF), a fluoroelastomer, a polyester, a polyamide, polyethylene, polypropylene, polyacetal, polyetherketones, polyarylketones, polyether sulfones, polyphenyl sulfones, polysulfones, polyarylsulfones, polyetherimides, polyimides, poly(amide-imides), PVC, PVDC-PVC copolymers, CPVC, polyfurans, poly(phenylene sulfides), epoxy resins, silicone elastomers, nitrile rubbers, chloroprene polymers, chlorosulfonated rubbers, polysulfide rubbers, ethylene-polypropylene elastomers, butyl rubbers, polyacrylic rubbers, a fiber-reinforced plastic, glass, and other suitable electrically insulative, chemically compatible materials. As yet another alternative, the insulating material 52 may be comprised of an epoxy material, such as aliphatic epoxy, chemically resistant thermoplastic hot melt materials, polyamide, polyester, polyurethane, epoxy, polyethylene-vinyl acetate, UV curable resin, acrylate, methacrylate, thermosetting resin, aromatic epoxy, silicone, a thermally cured adhesive, and a UV-cured adhesive. Such materials may be applied by spraying, brushing, dipping, vapor deposition, etc. A curing process may or may not be required depending upon the particular materials used. The insulating material 52 may also be comprised of a ceramic material. This product may also be applied by spraying, brushing, dipping, vapor deposition, etc., and it may be subsequently cured.

The insulating material 52 will assist in providing the desired degree of electrical isolation of the various components of the implantable medical device. Moreover, as will be recognized by those skilled in the art after a complete reading of the present application, the insulating material 52 of the present invention may be used in lieu of, or in addition to, other isolation methods and devices commonly employed with modern implantable medical devices. Moreover, the insulating material 52 need not be formed on all of the interior surfaces and/or exterior surfaces of the component case 50 and/or device housing 34. For example, the insulating material 52 may be formed on the interior surfaces of the cavity 47 in the component case bottom 42, while the bottom surface 45 of the component case top 44 is not lined. In that case, a plastic liner, such as the liner top 16 depicted in FIG. 1, may be positioned over the component 40 before or after the component 40 is positioned in the cavity 47 of the component case bottom 42. However, in some embodiments, the insulating material 52 is formed on all of the interior surfaces and/or exterior surfaces of the component case 50 and/or device housing 34. Lastly, the insulating material 52 formed on the component case 50 and/or device housing 34 may be comprised of different materials.

In general, the present invention is directed to various embodiments of an implantable medical device having a device housing and/or component case with an insulation material or layer formed thereon. In one illustrative embodiment, the device comprises a device housing and a component case, each of which have a plurality of interior surfaces and exterior surfaces, and an insulating material or layer formed on at least one of the interior surfaces and/or exterior surfaces of the device housing and/or the component case. In some embodiments, the insulating material is formed on at least some of the interior surfaces and/or exterior surfaces of both the device housing and component case. In even further embodiments, the insulating material or layer may be formed on all of the interior surfaces and/or exterior surfaces of the component case and/or the device housing. The insulating material may be comprised of a metal oxide, e.g., aluminum oxide, titanium oxide, etc., a plastic material, an epoxy material, or a ceramic material.

The present invention is also directed to various methods for forming the inventive medical devices disclosed herein. In one embodiment, the method comprises providing at least one of a device housing and a component case, each of which have a plurality of interior surfaces and/or exterior surfaces, and forming an insulating material or layer on at least one of the interior surfaces and/or exterior surfaces of the device housing and/or the component case. In some embodiments, the method involves forming the insulating material on at least some of the interior surfaces and/or exterior surfaces of both the device housing and the component case. In even further embodiments, the method involves forming the insulating material or layer on all of the interior surfaces and/or exterior surfaces of the device housing and/or component case. In some embodiments, the method involves forming an insulating material or layer comprised of a metal oxide, a plastic material, an epoxy material or a ceramic material. In yet further embodiments, the insulating material is comprised of a metal oxide that is formed by performing at least one oxidation process. The insulating material or layer may be comprised of a plastic, an epoxy, or a ceramic material, and it may be formed by a spraying or brushing process followed by, in some cases, a curing process.

The particular embodiments disclosed above are illustrative only, as the invention may be modified and practiced in different but equivalent manners apparent to those skilled in the art having the benefit of the teachings herein. For example, an alternative embodiment includes a device housing adapted to directly receive a component. In that embodiment, a component case is not necessarily required, and at least one surface of the device housing includes an insulating layer formed thereon. In another example, the process steps set forth above may be performed in a different order. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed:

1. An implantable medical device, comprising:
a device housing, said device housing having a plurality of interior surfaces;
a component case positioned within said device housing, said component case having a plurality of interior surfaces; and
an insulating material formed on at least one of said plurality of interior surfaces of at least one of said device housing and said component case.

2. The implantable medical device of claim 1, further comprising at least one of a capacitor, a battery, a fuel cell and an electronics module positioned within at least one of said device housing and said component case.

3. The implantable medical device of claim 1, wherein said device housing is comprised of at least one of aluminum, titanium and stainless steel.

4. The implantable medical device of claim 1, wherein said component case is comprised of at least one of aluminum, titanium and stainless steel.

5. The implantable medical device of claim 1, wherein said insulating material is formed on all of said interior surfaces of said component case.

6. The implantable medical device of claim 1, wherein said insulating material is formed on all of said interior surfaces of said device housing.

7. The implantable medical device of claim 1, wherein said insulating material is formed on at least one of said plurality of interior surfaces of each of said device housing and said component case.

8. The implantable medical device of claim 1, wherein said insulating material is formed on all of said interior surfaces of said device housing and said component case.

9. The implantable medical device of claim 1, wherein said insulating material is comprised of a metal oxide.

10. The implantable medical device of claim 1, wherein said insulating material has a thickness ranging from approximately 10 nm-500 μm.

11. The implantable medical device of claim 1, wherein at least one of said device housing and said component case is comprised of a metal and said insulating material is comprised of a metal oxide.

12. The implantable medical device of claim 1, wherein at least one of said device housing and said component case is comprised of aluminum and said insulating material is comprised of aluminum oxide.

13. The implantable medical device of claim 1, wherein at least one of said device housing and said component case is comprised of titanium and said insulating material is comprised of titanium oxide.

14. The implantable medical device of claim 1, wherein said insulating material is comprised of a plastic material.

15. The implantable medical device of claim 1, wherein said insulating material is selected from the group consisting of a fluoroplastic, a fluoroelastomer, a polyester, a polyamide, polyethylene, polypropylene, polyacetal, polyetherketones, polyarylketones, polyether sulfones, polyphenyl sulfones, polysulfones, polyarylsulfones, polyetherimides, polyimides, poly(amide-imides), PVC, PVDC-PVC copolymers, CPVC, polyfurans, poly(phenylene sulfiles), epoxy resins, silicone elastomers, nitrile rubbers, chloroprene polymers, chlorosulfonated rubbers, polysulfide rubbers, ethylene-polypropylene elastomers, butyl rubbers, polyacrylic rubbers, a fiber-reinforced plastic, and glass.

16. The implantable medical device of claim 1, wherein said insulating material is comprised of an epoxy material.

17. The implantable medical device of claim 1, wherein said insulating material is selected from the group consisting of aliphatic epoxy, chemically resistant thermoplastic hot melt materials, polyamide, polyester, polyurethane, epoxy, polyethylene-vinyl acetate, UV curable resin, acrylate, methacrylate, thermosetting resin, aromatic epoxy, silicone, a thermally cured adhesive, and a UV-cured adhesive.

18. The implantable medical device of claim 1, wherein said insulating material is comprised of a ceramic material.

19. The implantable medical device of claim 1, further comprising an insulating liner positioned between a component comprised of at least one of a capacitor, a battery, a fuel cell and an electronics module positioned in said component case and an insulating material formed on at least one interior surface of said component case.

20. The implantable medical device of claim 8, wherein said insulating material is comprised of at least one of a metal oxide, a plastic material, an epoxy material and a ceramic material.

21. The implantable medical device of claim 1, wherein said device housing further comprises a plurality of exterior surfaces and said insulating material is formed on at least one of said exterior surfaces of said device housing.

22. The implantable medical device of claim 1, wherein said component case further comprises a plurality of exterior surfaces and said insulating material is formed on at least one of said exterior surfaces of said component case.

23. The implantable medical device of claim 21, wherein said component case further comprises a plurality of exterior surfaces and said insulating material is formed on at least one of said exterior surfaces of said component case.

24. The implantable medical device of claim 7, wherein said component case further comprises a plurality of exterior surfaces and said insulating material is formed on at least one of said exterior surfaces of said component case.

25. The implantable medical device of claim 24, wherein said device housing further comprises a plurality of exterior surfaces and said insulating material is formed on at least one of said exterior surfaces of said device housing.

26. An implantable medical device, comprising:
a device housing, said device housing having a plurality of exterior surfaces;
a component case positioned within said device housing, said component case having a plurality of exterior surfaces; and
an insulating material formed on at least one of said plurality of exterior surfaces of at least one of said device housing and said component case.

27. The implantable medical device of claim 26, further comprising at least one of a capacitor, a battery, a fuel cell and an electronics module positioned within at least one of said device housing and said component case.

28. The implantable medical device of claim 26, wherein said device housing is comprised of at least one of aluminum, titanium and stainless steel.

29. The implantable medical device of claim 26, wherein said component case is comprised of at least one of aluminum, titanium and stainless steel.

30. The implantable medical device of claim 26, wherein said insulating material is formed on all of said exterior surfaces of said component case.

31. The implantable medical device of claim 26, wherein said insulating material is formed on all of said exterior surfaces of said device housing.

32. The implantable medical device of claim 26, wherein said insulating material is formed on at least one of said plurality of exterior surfaces of said device housing and said component case.

33. The implantable medical device of claim 26, wherein said insulating material is formed on all of said exterior surfaces of said device housing and said component case.

34. The implantable medical device of claim 26, wherein said insulating material is comprised of a metal oxide.

35. The implantable medical device of claim 26, wherein said insulating material has a thickness ranging from approximately 10 nm-500 μm.

36. The implantable medical device of claim 26, wherein at least one of said device housing and said component case is comprised of a metal and said insulating material is comprised of a metal oxide.

37. The implantable medical device of claim 26, wherein at least one of said device housing and said component case is comprised of aluminum and said insulating material is comprised of aluminum oxide.

38. The implantable medical device of claim 26, wherein at least one of said device housing and said component case is comprised of titanium and said insulating material is comprised of titanium oxide.

39. The implantable medical device of claim 26, wherein said insulating material is comprised of a plastic material.

40. The implantable medical device of claim 26, wherein said insulating material is selected from the group consisting of a fluoroplastic, a fluoroelastomer, a polyester, a polyamide, polyethylene, polypropylene, polyacetal, polyetherketones, polyarylketones, polyether sulfones, polyphenyl sulfones, polysulfones, polyarylsulfones, polyetherimides, polyimides, poly(amide-imides), PVC, PVDC-PVC copolymers, CPVC, polyfurans, poly(phenylene sulfiles), epoxy resins, silicone elastomers, nitrile rubbers, chloroprene polymers, chlorosulfonated rubbers, polysulfide rubbers, ethylene-polypropylene elastomers, butyl rubbers, polyacrylic rubbers, a fiber-reinforced plastic and glass.

41. The implantable medical device of claim 26, wherein said insulating material is comprised of an epoxy material.

42. The implantable medical device of claim 26, wherein said insulating material is selected from the group consisting of aliphatic epoxy, chemically resistant thermoplastic hot melt materials, polyamide, polyester, polyurethane, epoxy, polyethylene-vinyl acetate, UV curable resin, acrylate, methacrylate, thermosetting resin, aromatic epoxy, silicone, a thermally cured adhesive, and a UV-cured adhesive.

43. The implantable medical device of claim 26, wherein said insulating material is comprised of a ceramic material.

44. The implantable medical device of claim 26, further comprising an insulating liner positioned between a component comprised of at least one of a capacitor, a battery, a fuel cell and an electronics module positioned in said component case and said insulating material formed on said at least one interior surface of said component case.

45. The implantable medical device of claim 33, wherein said insulating material is comprised of at least one of a metal oxide, a plastic material, an epoxy material and a ceramic material.

46. The implantable medical device of claim 26, wherein said device housing further comprises a plurality of interior surfaces and said insulating material is formed on at least one of said interior surfaces of said device housing.

47. The implantable medical device of claim 26, wherein said component case further comprises a plurality of interior surfaces and said insulating material is formed on at least one of said interior surfaces of said component case.

48. The implantable medical device of claim 46, wherein said component case further comprises a plurality of interior surfaces and said insulating material is formed on at least one of said interior surfaces of said component case.

49. The implantable medical device of claim 32, wherein said component case further comprises a plurality of interior surfaces and said insulating material is formed on at least one of said interior surfaces of said component case.

50. The implantable medical device of claim 49, wherein said device housing further comprises a plurality of exterior surfaces and said insulating material is formed on at least one of said exterior surfaces of said device housing.

51. A method, comprising:
providing at least one of a device housing for an implantable medical device and a component case adapted to have a component comprised of at least one of a capacitor module, a battery, a fuel cell and an electronics module positioned therein, each of said device housing and said component case having a plurality of interior surfaces; and
forming an insulating material on at least one of said interior surfaces of at least one of said device housing and said component case.

52. The method of claim 51, wherein forming an insulating material comprises forming an insulating material comprised of a metal oxide.

53. The method of claim 51, wherein forming an insulating material comprises forming an insulating material comprised of at least one of an aluminum oxide and a titanium oxide.

54. The method of claim 51, wherein forming an insulating material comprises forming an insulating material having a thickness ranging from approximately 10 nm-500 μm.

55. The method of claim 51, wherein forming an insulating material comprises forming an insulating material comprised of a plastic material.

56. The method of claim 51, wherein forming an insulating material comprises forming an insulating material selected from the group consisting of a fluoroplastic, a fluoroelastomer, a polyester, a polyamide, polyethylene, polypropylene, polyacetal, polyetherketones, polyarylketones, polyether sulfones, polyphenyl sulfones, polysulfones, polyarylsulfones, polyetherimides, polyimides, poly(amide-imides), PVC, PVDC-PVC copolymers, CPVC, polyfurans, poly(phenylene sulfiles), epoxy resins, silicone elastomers, nitrile rubbers, chloroprene polymers, chlorosulfonated rubbers, polysulfide rubbers, ethylene-polypropylene elastomers, butyl rubbers, polyacrylic rubbers, a fiber-reinforced plastic and glass.

57. The method of claim 51, wherein forming an insulating material comprises forming an insulating material comprised of an epoxy material.

58. The method of claim 51, wherein forming an insulating material comprises forming an insulating material selected from the group consisting of aliphatic epoxy, chemically resistant thermoplastic hot melt materials, polyamide, polyester, polyurethane, epoxy, polyethylene-vinyl acetate, UV curable resin, acrylate, methacrylate, thermosetting resin, aromatic epoxy, silicone, a thermally cured adhesive, and a UV-cured adhesive.

59. The method of claim 51, wherein forming an insulating material comprises forming an insulating material comprised of a ceramic material.

60. The method of claim 51, wherein forming an insulating material comprises performing at least one oxidation process to form an insulating material comprised of a metal oxide.

61. The method of claim 51, wherein forming an insulating material on at least one of said interior surfaces of at least one of said device housing and said component case comprises forming an insulating material on at least one of said interior surfaces of said device housing and on at least one of said interior surfaces of said component case.

62. The method of claim 51, further comprising positioning said component in said component case.

63. The method of claim 51, further comprising positioning an insulating liner around said component prior to positioning said component in said component case.

64. The method of claim 51, wherein said device housing and said component case further comprise a plurality of exterior surfaces and wherein said method further comprises forming said insulating material on at least one of said exterior surfaces of at least one of said device housing and component case.

65. The method of claim 61, wherein said device housing and said component case further comprise a plurality of exterior surfaces and wherein said method further comprises forming said insulating material on at least one of said exterior surfaces of at least one of said device housing and component case.

66. The method of claim 61, wherein said device housing and said component case further comprise a plurality of exterior surfaces and wherein said method further comprises forming said insulating material on at least one of said exterior surfaces of said device housing and on at last one of said exterior surfaces of said component case.

67. A method, comprising:
providing at least one of a device housing for an implantable medical device and a component case adapted to have a component comprised of at least one of a capacitor module, a battery, a fuel cell and an electronics module positioned therein, each of said device housing and said component case having a plurality of exterior surfaces; and
forming an insulating material on at least one of said exterior surfaces of at least one of said device housing and said component case.

68. The method of claim 67, wherein forming an insulating material comprises forming an insulating material comprised of a metal oxide.

69. The method of claim 67, wherein forming an insulating material comprises forming an insulating material comprised of at least one of an aluminum oxide and a titanium oxide.

70. The method of claim 67, wherein forming an insulating material comprises forming an insulating material having a thickness ranging from approximately 10 nm-500 μm.

71. The method of claim 67, wherein forming an insulating material comprises forming an insulating material comprised of a plastic material.

72. The method of claim 67, wherein forming an insulating material comprises forming an insulating material selected from the group consisting of a fluoroplastic, a fluoroelastomer, a polyester, a polyamide, polyethylene, polypropylene, polyacetal, polyetherketones, polyarylketones, polyether sulfones, polyphenyl sulfones, polysulfones, polyarylsulfones, polyetherimides, polyimides, poly(amide-imides), PVC, PVDC-PVC copolymers, CPVC, polyfurans, poly(phenylene sulfiles), epoxy resins, silicone elastomers, nitrile rubbers, chloroprene polymers, chlorosulfonated rubbers, polysulfide rubbers, ethylene-polypropylene elastomers, butyl rubbers, polyacrylic rubbers, a fiber-reinforced plastic and glass.

73. The method of claim 67, wherein forming an insulating material comprises forming an insulating material comprised of an epoxy material.

74. The method of claim 67, wherein forming an insulating material comprises forming an insulating material selected from the group consisting of aliphatic epoxy, chemically resistant thermoplastic hot melt materials, polyamide, polyester, polyurethane, epoxy, polyethylene-vinyl acetate, UV curable resin, acrylate, methacrylate, thermosetting resin, aromatic epoxy, silicone, a thermally cured adhesive, and a UV-cured adhesive.

75. The method of claim 67, wherein forming an insulating material comprises forming an insulating material comprised of a ceramic material.

76. The method of claim 67, wherein forming an insulating material comprises performing at least one oxidation process to form an insulating material comprised of a metal oxide.

77. The method of claim 67, wherein forming an insulating material on at least one of said exterior surfaces of at least one of said device housing and said component case comprises forming an insulating material on at least one of said exterior surfaces of said device housing and on at least one of said exterior surfaces of said component case.

78. The method of claim 67, further comprising positioning said component in said component case.

79. The method of claim 67, further comprising positioning an insulating liner around said component prior to positioning said component in said component case.

80. The method of claim 67, wherein said device housing and said component case further comprise a plurality of interior surfaces and wherein said method further comprises forming said insulating material on at least one of said interior surfaces of at least one of said device housing and component case.

81. The method of claim 77, wherein said device housing and said component case further comprise a plurality of interior surfaces and wherein said method further comprises forming said insulating material on at least one of said interior surfaces of at least one of said device housing and component case.

82. The method of claim 77, wherein said device housing and said component case further comprise a plurality of interior surfaces and wherein said method further comprises forming said insulating material on at least one of said interior surfaces of said device housing and on at least one of said interior surfaces of said component case.

83. An implantable medical device, comprising:
a device housing, said device housing having a plurality of surfaces; and
an insulating material formed on at least one of said plurality surfaces.

84. The implantable medical device of claim 83, further comprising at least one of a capacitor, a battery, a fuel cell and an electronics module positioned within said device housing.

85. The implantable medical device of claim 83, wherein said device housing is comprised of at least one of aluminum, titanium and stainless steel.

86. The implantable medical device of claim 83, wherein said insulating material is formed on all of said interior surfaces of said device housing.

87. The implantable medical device of claim 83, wherein said insulating material is formed on at least one exterior surface of said device housing.

88. The implantable medical device of claim 83, wherein said insulating material is comprised of a metal oxide.

89. The implantable medical device of claim 83, wherein said insulating material has a thickness ranging from approximately 10 nm-500 μm.

90. The implantable medical device of claim 83, wherein said device housing is comprised of a metal and said insulating material is comprised of a metal oxide.

91. The implantable medical device of claim 83, wherein said insulating material is comprised of a plastic material.

92. The implantable medical device of claim 83, wherein said insulating material is selected from the group consisting of a fluoroplastic, a fluoroelastomer, a polyester, a polyamide, polyethylene, polypropylene, polyacetal, polyetherketones, polyarylketones, polyether sulfones, polyphenyl sulfones, polysulfones, polyarylsulfones, polyetherimides, polyimides, poly(amide-imides), PVC, PVDC-PVC copolymers, CPVC, polyfurans, poly(phenylene sulfiles), epoxy resins, silicone elastomers, nitrile rubbers, chloroprene polymers, chlorosulfonated rubbers, polysulfide rubbers, ethylene-polypropylene elastomers, butyl rubbers, polyacrylic rubbers, a fiber-reinforced plastic, and glass.

93. The implantable medical device of claim 83, wherein said insulating material is comprised of an epoxy material.

94. The implantable medical device of claim 83, wherein said insulating material is selected from the group consisting of aliphatic epoxy, chemically resistant thermoplastic hot melt materials, polyamide, polyester, polyurethane, epoxy, polyethylene-vinyl acetate, UV curable resin, acrylate, methacrylate, thermosetting resin, aromatic epoxy, silicone, a thermally cured adhesive, and a UV-cured adhesive.

95. The implantable medical device of claim 83, wherein said insulating material is comprised of a ceramic material.
